(19) United States
(12) Patent Application Publication
(10) Pub. No.: US 2016/0229778 A1
(43) Pub. Date: Aug. 11, 2016

(54) DIRECT B-ARYLATION OF CARBONYL COMPOUNDS

(71) Applicant: Board of Regents, The University of Texas System, Austin, TX (US)

(72) Inventors: Guangbin Dong, Austin, TX (US); Zhongxing Huang, Austin, TX (US)

(21) Appl. No.: 14/535,969

(22) Filed: Nov. 7, 2014

Related U.S. Application Data
(60) Provisional application No. 61/901,882, filed on Nov. 8, 2013.

(51) Int. Cl. C07C 45/68; C07C 253/30; C07C 201/12; C07D 211/74; C07C 259/00; C07C 67/293; C07D 333/22; C07D 295/26; C07C 231/12

(52) U.S. Cl. CPC C07C 45/68; C07D 295/26; C07C 253/30; C07C 201/12; C07C 231/12; C07C 259/00; C07C 67/293; C07D 333/22; C07D 211/74; C07C 2101/14; C07C 2101/18; C07C 2101/08

(57) ABSTRACT

Disclosed is a method for the β-C—H H functionalization of carbonyl compounds that is both selective and broadly applicable. The methods provide direct β-arylation of carbonyl compound with a diverse array of aryl or heteroaryl halides, aryl or heteroryl tosylate, aryl or heteroaryl triflates, or diaryliodonium salts, by palladium catalysis in the presence of a ligand and promoter.

### CROSS REFERENCE TO RELATED APPLICATIONS

This application claims the benefit of priority to U.S. Provisional Application 61/901,882, filed Nov. 8, 2013, which is incorporated by reference herein in its entirety.

### BACKGROUND

Functionalization and transformation of carbonyl compounds are essential to organic synthesis. While the a-position of the carbonyl group can be readily functionalized through enolate chemistry, the corresponding β-C—H bonds are, typically, not reactive. As an important class of carbonyl compound-functionalization reactions, arylation (substitution of C—H bonds with aryl groups) has been achieved at the α-position through palladium-catalyzed couplings of carbonyl compounds and aryl halides (FIG. 1a; Palucki, et al., Palladium-catalyzed α-arylation of ketones. J. Am. Chem. Soc. 119, 11108-11109 (1997); Hamann, et al., Palladium-catalyzed direct α-arylation of ketones. Rate acceleration by sterically hindered chelating ligands and reductive elimination from a transition metal enolate complex. J. Am. Chem. Soc. 119, 12382-12383 (1997); Satoh, et al., Palladium-catalyzed regioselective mono- and diarylation reactions of 2-phenylphenols and naphthols with aryl halides. Angew. Chem. Int. Ed. 36, 1740-1742 (1997)). The resulting structural motifs are frequently found in pharmaceutical, materials and agrochemical products (Johansson, et al., Metal-catalyzed α-arylation of carbonyl and related molecules: novel trends in C—C bond formation by C—H bond functionalization. Angew. Chem. Int. Ed. 49, 676-707 (2010); Bellina, et al., Transition metal-catalyzed direct arylation of substrates with activated sp³-hybridized C—H bonds and some of their synthetic equivalents with aryl halides and pseudohalides. Chem. Rev. 110, 1082-1146 (2010)). The practicality and wide applicability of the α-arylation reactions are likely attributed to the use of readily available starting materials (i.e., simple carbonyl compounds, aryl halides), the efficient and tunable palladium catalysts, the scalable reaction conditions, and high functional group tolerance. However, the corresponding β-arylation, while being equally important, has remained largely underdeveloped.

β-aryl-substituted carbonyl compounds are synthesized through conjugate addition of aryl (often metal-based) nucleophiles to α,β-unsaturated carbonyl compounds (FIG. 1b; Rossiter, et al., Asymmetric conjugate addition. Chem. Rev. 92, 771-806 (1992); Gutnov, Palladium-catalyzed asymmetric conjugate addition of aryl-metal species. Eur. J. Org. Chem. 4547-4554 (2008); Hayashi, et al., Rhodium-catalyzed asymmetric 1,4-addition and its related asymmetric reactions. Chem. Rev. 103, 2829-2844 (2003)); however, both reactants require additional synthetic steps and redox processes to prepare. For example, the conjugated enones are usually prepared in 1-3 steps from the corresponding saturated ketones via direct or indirect dehydrogenation (an oxidation process to generate electrophiles), and most aryl nucleophiles (e.g., organo-cuprate or boron reagents) ultimately come from the corresponding aryl halides through metallation reactions (a reduction process to generate nucleophiles). To circumvent this drawback, elegant directing group-based strategies were developed enabling the direct β-arylation, albeit limited to linear ester and amide substrates (Jorgensen, et al., Efficient synthesis of α-aryl esters by room-temperature palladium-catalyzed coupling of aryl halides with ester enolates J. Am. Chem. Soc. 124, 12557-12565 (2002); Zaitsev, et al., Highly regioselective arylation of sp³ C—H bonds catalyzed by palladium acetate. J. Am. Chem. Soc. 127, 13154-13155 (2005); Shabashov, et al., Auxiliary-assisted palladium-catalyzed arylation and alkylation of sp² and sp³ carbon-hydrogen bonds. J. Am. Chem. Soc. 132, 3965-3972 (2010); Reddy, et al., Novel acetoxylation and C—C coupling reactions at unactivated positions in a-amino acid derivatives. Org. Lett. 8, 3391-3394 (2006); Wang, et al., Pd(II)-Catalyzed cross-coupling of sp³ C—H bonds with sp² and sp³ boronic acids using air as the oxidant. J. Am. Chem. Soc. 130, 7190-7191 (2008); Wasa, et al., Pd(0)/PR₃-catalyzed intermolecular arylation of sp³ C—H bonds. J. Am. Chem. Soc. 131, 9886-9887 (2009); Wasa, et al. Ligand-enabled methylene C(sp³)—H bond activation with a Pd(II) catalyst. J. Am. Chem. Soc. 134, 18570-18572 (2012); Renaudat, et al. Palladium-catalyzed β-arylation of carboxylic esters. Angew. Chem. Int. Ed. 49, 7261-7265 (2010); Shang, et al., β-Arylation of carboxamides via iron-catalyzed C(sp³)—H bond activation. J. Am. Chem. Soc. 135, 6030-6032 (2013)). Recently, MacMillan and coworkers reported a photoredox-based β-arylation of aldehydes and ketones, using electron-deficient arylnitriles as the coupling partner (Pirnot, et al., Photoredox activation for the direct β-arylation of ketones and aldehydes. Science 339, 1593-1596 (2013)).

A general solution to the β-arylation problem possessing the broadly applicable feature of Buchwald-Hartwig-Miura α-arylation, which includes the direct use of readily accessible substrates, high functional group compatibility, and scalability, is needed. The methods and compositions address these and other needs.

### SUMMARY

The subject matter disclosed herein relates to methods of making compositions and the compositions themselves. In particular, the subject matter disclosed herein generally relates to methods of functionalizing a carbonyl compound at a β-carbon with an aryl or heteroaryl group. The disclosed methods involve a catalytic coupling between carbonyls with a β-carbon and aryl or heteroaryl halides/tosylates/triflates or diaryliodonium salts, many of which are widely available. The disclosed methods exhibit site-selectivity at the β-position of carbonyls rather than the α-position.

Additional advantages of the disclosed subject matter will be set forth in part in the description that follows and the Figures, and in part will be obvious from the description, or can be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIG. 1 is a group of schematics showing differences among α- and β-arylation of ketones. FIG. 1a is a schematic of a Buchwald-Hartwig-Miura α-arylation. Ar=aryl group; L=neutral ligand; X=anionic ligand. FIG. 1b is a schematic of a β-arylation of carbonyl compounds. FIG. 1c is a catalytic cycle of a Pd-catalyzed direct β-arylation of ketones.

FIG. 2 is a table that shows the effects of various conditions on the arylation of cyclohexanone with iodobenzene as model reactants. Ph=phenyl group; Me=methyl; Cy=cyclohexyl; Bu=butyl; Pr=propyl; Ac=acetate.

FIG. 3 is a table that exemplifies the diversity of substrates that can be used in the Pd-catalyzed β-arylation with aryl iodides. Reaction conditions: aryl iodide (0.4 mmol), ketone (1.0 mmol), Pd(TFA)₂ (0.04 mmol), P(i-Pr)₃ (0.08 mmol), AgTFA (0.8 mmol), HFIP (1 mL), dioxane (1 mL), 80° C., 12 hours. *1.0 equiv. of the ketone and 2.5 equiv. of iodobenzene were used. †5.0 equiv. of the ketone was used. ‡10.0 equiv. of the ketone was used.

FIG. 4a is a schematic showing the use of aryl bromides as substrates. FIG. 4b is a schematic showing gram-scale reaction of the Pd-catalyzed β-arylation of ketones. FIG. 4c is a schematic showing substitution of the silver salts with copper and potassium salts.

FIG. 5 is a schematic showing the Pd-catalyzed β-arylation of 4-phenylcyclohexanone.

FIG. 6 is a table that exemplifies the diversity of various aryl sources and ligands that can be used in the Pd-catalyzed β-acylation.

FIG. 7 is a schematic showing the synthesis of ligand L1 and its stereoisomers.

FIG. 8 is a table that exemplifies the diversity of substrates that can be used in the Pd-catalyzed β-arylation with mesitylaryliodonium salts.

FIG. 9 is a table that exemplifies the diversity of carbonyl compounds that can be used in the Pd-catalyzed β-arylation with mesitylaryliodonium salts.

FIG. 10 is a schematic showing the synthesis of ligands, L9, L10, and L11, and a generation Pd-catalyzed β-arylation reaction with L9.

### DETAILED DESCRIPTION

The materials, compounds, compositions, articles, and methods described herein may be understood more readily by reference to the following detailed description of specific aspects of the disclosed subject matter and the Examples and Figures included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, reference to “an agent” includes mixture of two or more such agents, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect.

### Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below. Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OZ¹ where Z¹ is alkyl as defined above.

The term “alkenyl” as used herein is a hydrocarbon group of from 2 to 24 carbon atoms with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” or “CO” is a short hand notation for C═O, which is also refered to herein as a “carbonyl.”

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” as used herein refers to the fluorine, chlorine, bromine, and iodine. The corresponding term “halo”, e.g., fluoro, chloro, bromo, and iodo as used herein refer to the corresponding halogen radical or ion.

The term “hydroxyl” as used herein is represented by the formula —OH.

The term “cyano” as used herein is represented by the formula —CN. Cyanide is used to refer to the cyanide ion CN⁻.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

The term “triflate” or “Tf” as used herein is represented by the formula CF₃SO₃—

The term “tosylate” or “Ts” as used herein is represented by the formula CH₃PhSO₃—.

The term “mesityl” or “mes” is 1,3,5-trimethylphenyl.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

### Methods

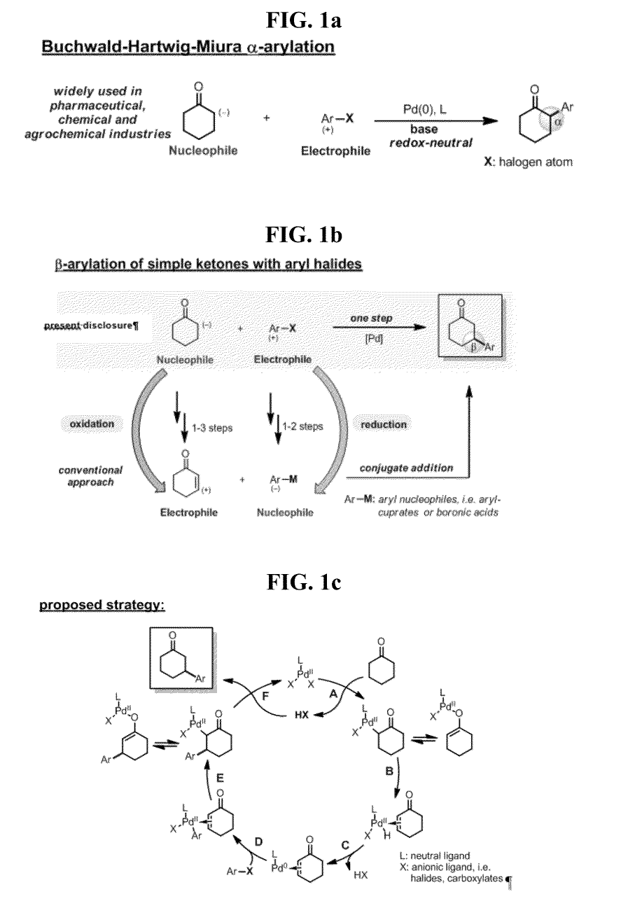

Disclosed herein are methods of functionalizing a carbonyl compound at a β-carbon with an aryl or heteroaryl group. In the disclosed methods a carbonyl compound comprising a carbon β to the carbonyl (i.e., a β-carbon) is contacted with an aryl or heteroaryl halide, aryl or heteroaryl tosylate, aryl or heteroaryl triflate, or diaryliodonium salt, a palladium catalyst, a ligand and a promoter. The disclosed methods couple ketone dehydrogenation, aryl or heteroaryl-halide/tosylate/triflate/iodonium activation, and conjugate addition by taking advantage of different reactive modes of palladium complexes (FIG. 1c). Palladium-mediated ketone dehydrogenation is known to provide a straightforward route to activate the C—H bond β to the carbonyl (Theissen, A new method for the preparation of α,β-unsaturated carbonyl compounds. J. Org. Chem. 36, 752-757 (1971)), which follows a sequence of Pd(II)-enolate formation, β-H elimination and reductive elimination of an acid (HX) to give an enone and Pd(0) species (Steps A-C, FIG. 1c). For example, an efficient catalytic process was recently reported by Stahl and Diao by employing oxygen gas to oxidize the Pd(0) intermediate to regenerate the active Pd(II) catalyst (Diao, et al., Synthesis of cyclic enones via direct palladium-catalyzed aerobic dehydrogenation of ketones. J. Am. Chem. Soc. 133, 14566-14569 (2011); Diao, et al., Direct aerobic α,β-dehydrogenation of aldehydes and ketones with a Pd(TFA)₂/4,5-diazafluorenone catalyst. Chem. Sci. 3, 887-891 (2012)).

In the disclosed methods, aryl or heteroaryl halides/tosylates/triflates or diaryliodonium salts serve as a stoichiometric oxidant to promote ketone oxidation to conjugated enones as the oxidative addition of Pd(0) into aryl or heteroaryl-halide/tosylate/triflate bonds to give Pd(II) is a well-established process (Step D). The resulting aryl or heteroaryl-Pd(II) species undergoes migratory insertion into the conjugated enone olefin to provide a Pd(II)-enolate (Gutnov, Eur. J. Org. Chem. 4547-4554 (2008)) (Step E), which upon protonation by HX would give the β-arylated product and regenerate the Pd(II) catalyst (Step F). While not wishing to be bound by theory, it is believe that a key feature of the disclosed method is that ketone oxidation and conjugate addition are incorporated into a single catalytic cycle by using aryl or heteroaryl halides/tosylates/triflates/iodiniums as both the oxidant and aryl or heteroaryl source. Comparing to the conventional 1,4-addition approach (FIG. 1b), the disclosed methods can, in many instances, streamline the synthesis of β-aryl or heteroaryl carbonyl compounds by reducing the number of steps and/or oxidation/reduction operations (for examples of a tandem ketone oxidation followed by addition of a nucleophile see Hayashi, et al., Oxidative and enantioselective cross-coupling of aldehydes and nitromethane catalyzed by diphenylprolinol silyl ether. Angew. Chem. Int. Ed. 50, 3920-3924 (2011); Zhang, et al. Organocatalytic enantioselective β-functionalization of aldehydes by oxidation of enamines and their application in cascade reactions. Nat. Commun. 2, 211-217 (2011); Leskinen, et al., Palladium-catalyzed dehydrogenative β′-functionalization of β-keto esters with indoles at room temperature. J. Am. Chem. Soc. 134, 5750-5753 (2012); Yip, et al., Palladium-catalyzed dehydrogenative Varylation of β′-arylation of β-keto esters under aerobic conditions: interplay of metal and Bronsted acids. Chem. Eur. J. 18, 12590-12594 (2012); for examples of N-hereocyclic carbene-catalyzed β-activation of linear aldehydes and esters and subsequent carbonyl transformations, see Mo, et al., Direct β-activation of saturated aldehydes to formal Michael acceptors through oxidative NHC catalysis. Angew. Chem. Int. Ed. 52, 8588-8591 (2013); Fu, et al., β-Carbon activation of saturated carboxylic esters through N-heterocyclic carbene organocatalysis. Nature Chem. Advance online publication, 21 Jul. 2013 (DOI 10.1038/NCHEM.1710)).

The success of the disclosed methods involved overcoming the following key challenges. First, an electrophilic palladium complex is generally required for the ketone oxidation (Diao, et al., Aerobic dehydrogenation of cyclohexanone to cyclohexenone catalyzed by Pd(DMSO)₂(TFA)₂: evidence for ligand-controlled chemoselectivity. J. Am. Chem. Soc. 135, 8205-8212 (2013)) (Steps A and B) while oxidative addition into carbon-halide bonds (Step D) prefers electron-rich catalysts. Thus, developing a catalyst system that is able to accommodate both needs during the catalytic cycle is non-trivial.

Second, dimerization of aryl or heteroaryl halides (Hennings, et al., Palladium-catalyzed (Ullmann-type) homocoupling of aryl or heteroaryl halides: a convenient and general synthesis of symmetrical biaryls via inter- and intramolecular coupling reactions Org. Lett. 1, 1205-1208 (1999)) and over-oxidations to phenols and/or to β-aryl or heteroaryl enones (Ueno, et al., Nickel-catalyzed formation of a carbon-nitrogen bond at the β-position of saturated ketones. Angew. Chem. Int. Ed. 48, 4543-4545 (2009); Shang, et al. Pd-catalyzed C—H olefination of (hetero)arenes by using saturated ketones as an olefin source. Angew. Chem. Int. Ed. 52, 1299-1303 (2013); Moon, et al., Palladium-catalyzed dehydrogenation/oxidative cross-coupling sequence of β-heteroatom-substituted ketones. Angew. Chem. Int. Ed. 51, 11333-11336 (2012); Izawa, et al., Palladium-catalyzed aerobic dehydrogenation of substituted cyclohexanones to phenols. Science 333, 209-213 (2011); Pun, et al., Aerobic dehydrogenation of cyclohexanone to phenol catalyzed by Pd(TFA)₂/2-Dimethylaminopyridine: evidence for the role of Pd nanoparticles. J. Am. Chem. Soc. 135, 8213-8221 (2013)), which are known side reactions, must be inhibited under the reaction conditions.

Third, given that the Pd-mediated dehydrogenation works most efficiently with carboxylate ligands (Diao, J. Am. Chem. Soc. 135, 8205-8212 (2013); Ito, et al., Synthesis of α,β-unsaturated carbonyl compounds by palladium(II)-catalyzed dehydrosilylation of silyl enol ethers J. Org. Chem. 43, 1011-1013 (1978)), a suitable promoter must be found to extract halides from palladium and deliver carboxylate anions to restore the active catalyst.

The palladium-catalyzed direct β-arylation of carbonyl compounds disclosed herein overcomes several limitations and complements the scope of β-functionalization of carbonyl compounds. It has significant advantages over the conventional 1,4-addition methods, because this approach not only circumvents use of conjugated enones and aryl or heteroaryl nucleophiles as reactants, which need additional steps and redox procedures to prepare, but also tolerates base/nucleophile-sensitive moieties. It is distinct from the directing group-based C—H activation strategy by allowing both linear and cyclic substrates to react; direct coupling of the readily available aryl or heteroaryl halides/tosylates/triflates also distinguishes this work from the photo-redox β-arylation strategy. Furthermore, this methodology is scalable and chemoselective.

### Carbonyl Compounds

The disclosed methods are suitable for β-arylation of a diverse array of carbonyl compounds. As such the carbonyl compounds usable in the disclosed methods all comprise a carbon at the “β-position”. This carbon is illustrated in the following structure of a generic carbonyl compound suitable for use here; the dashed line indicates that the carbonyl compound can be cyclic or linear.

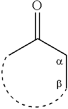

In certain examples of the disclosed methods, the carbonyl compound can be a linear carbonyl compound having Formula I or a cyclic carbonyl compound having Formula II.

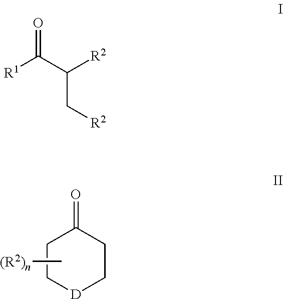

wherein

R¹ is OR³, N(R³)₂, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups;

n is 0, 1, 2, 3, 4, or 5;

each R² is, independent of any other, H, OH, SH, C(O)H, CO₂H, C(O)R³, CO₂R³, OR³, NO₂, CN, N(R³)₂, NC(O)R³, C(O)N(R³)₂, CNR³, Si(R³)₃, SO₂R³, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups; or

two R² groups are on adjacent carbons and together form a fused cycloalkyl ring, cycloheteroalkyl ring, aryl, or heteroaryl ring optionally substituted with one or more additional R² groups;

D is a bond, O, S, NR³, or —(C(R³)₂)₁₋₃; and

each R³ is, independent of any other, H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups.

In specific examples, the carbonyl compound can have Formula III:

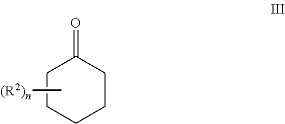

wherein R² and n are as defined above. In other examples, the carbonyl compound has Formula III wherein n is 0, i.e., cyclohexanone. In still other examples, the carbonyl compound has Formula III, where n is 1, and R² is substituted or unsubstituted C₁₋₂₀ alkyl, substituted or unsubstituted aryl, or substituted or unsubstituted heteroaryl.

### Aryl or Heteroaryl Halides, Aryl or Heteroaryl Tosylate, and Aryl or Heteroaryl Triflates

In the disclosed methods a carbonyl compound comprising a carbon β to the carbonyl (i.e., a β-carbon) can be contacted with an aryl or heteroaryl halide/tosylate/triflate. One advantage of these methods is that a diverse array of aryl or heteroaryl halides/tosylates/triflates can be used. For example, in the disclosed methods aryl or heteroaryl halide, aryl or heteroaryl tosylate, or aryl or heteroaryl triflate having Formula IV can be used.

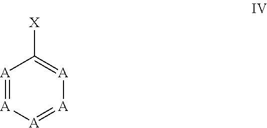

wherein

X is Cl, Br, I, OTf, or OTs;

each A is, independent of the others, N, CH, or CR⁴;

each R⁴ is, independent of the others, F, Cl, Br, I, OH, SH, C(O)H, CO₂H, C(O)R⁵, CO₂R⁵, OR⁵, NO₂, CN, N(R⁵)₂, NC(O)R⁵, C(O)N(R⁵)₂, CNR⁵, Si(R⁵)₃, SO₂R⁵, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups; or

two R⁴ groups are on adjacent carbons and together form a fused cycloalkyl ring, cycloheteroalkyl ring, aryl, or heteroaryl ring optionally substituted with one or more additional R⁴ groups; and

each R⁵ is, independent of any other, H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups.

In many examples, aryl or heteroaryl halides/tosylates/triflates of Formula IV can be used where X is Br or I. In other examples, X is OTs or OTf. In still other examples, at least on A in Formula IV is N. In still other examples, each R⁴ is substituted or unsubstituted C₁₋₆ alkyl, substituted or unsubstituted C₂₋₆ alkenyl, substituted or unsubstituted C₂₋₆ alkynyl, substituted or unsubstituted C₁₋₆ heteroalkyl, substituted or unsubstituted C₃₋₆ cycloalkyl, substituted or unsubstituted C₃₋₆ heterocycloalkyl, substituted or unsubstituted aryl, substituted or unsubstituted heteroaryl, or mixtures thereof.

In still other examples, aryl halides/tosylates/triflates of Formula V can be used.

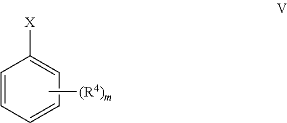

wherein X is Cl, Br, I, OTs, or OTf; m is 0, 1, 2, 3, 4, or 5; and R⁴ is as defined above. In specific examples, the aryl halide has Formula V where X is Br or I; and R⁴ is F, Cl, Br, I, OH, SH, C(O)H, CO₂H, C(O)R³, CO₂R³, OR³, NO₂, CN, N(R³)₂, NC(O)R³, C(O)N(R³)₂, CNR³, Si(R³)₃, SO₂R³, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups; and m is 1. In other examples, m is 2 or 3. In still other examples, R⁴ is F, Cl, Br, I, OH, SH, C(O)H, CO₂H, C(O)R⁵, CO₂R⁵, OR⁵, NO₂, CN, N(R⁵)₂, NC(O)R⁵, C(O)N(R⁵)₂, CNR⁵, Si(R⁵)₃, SO₂R⁵, substituted or unsubstituted C₁₋₂₀ alkyl, or substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups. In still other examples, each R⁴ is substituted or unsubstituted C₁₋₆ alkyl, substituted or unsubstituted C₂₋₆ alkenyl, substituted or unsubstituted C₂₋₆ alkynyl, substituted or unsubstituted C₁₋₆ heteroalkyl, substituted or unsubstituted C₃₋₆ cycloalkyl, substituted or unsubstituted C₃₋₆ heterocycloalkyl, substituted or unsubstituted aryl, substituted or unsubstituted heteroaryl, or mixtures thereof.

Stoichiometric amounts of the carbonyl compound and the aryl or heteroaryl halide are used in the disclosed methods.

### Diaryliodonium Salts

In the disclosed methods a carbonyl compound comprising a carbon β to the carbonyl (i.e., a β-carbon) can be contacted with a diaryliodonium salt. Diaryliodonium salts have high reactivity, stability, low-toxicity and are easy available. These aryl salts can be synthesized from a large collection of starting materials, including aryl iodide, aryl boronic acid, phenol or electron-rich arenes. In addition, the oxidative addition of the diaryliodonium salt to the Pd(0) would deliver an acetate or sulfonate ligand to the Pd center instead of a halide ligand in the case of aryl halide as the aryl source. Thus, the overall cost of the chemistry would be reduced significantly since no expensive heavy-metal salt is needed to scavenge the halide and restore the active catalyst. In specific examples, the disclosed methods can use a diaryliodonium salt having Formula VI.

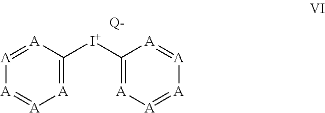

wherein

Q is a counter anion, such as F, Cl, Br, NO₂, HSO₄, HCO₃, OTs, OTf, and CH₃CO₂;

each A is, independent of the others, N, CH, or CR⁴;

each R⁴ is, independent of the others, F, Cl, Br, I, OH, SH, C(O)H, CO₂H, C(O)R⁵, CO₂R⁵, OR⁵, NO₂, CN, N(R⁵)₂, NC(O)R⁵, C(O)N(R⁵)₂, CNR⁵, Si(R⁵)₃, SO₂R⁵, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₁₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups; or

two R⁴ groups are on adjacent carbons and together form a fused cycloalkyl ring, cycloheteroalkyl ring, aryl, or heteroaryl ring optionally substituted with one or more additional R⁴ groups; and

each R⁵ is, independent of any other, H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups.

In many examples, diaryliodonium salts of Formula VI can be used where at least three A are CH₃. In still other examples, at least one A in Formula VI is N. In still other examples, each R⁴ is substituted or unsubstituted C₁₋₆ alkyl, substituted or unsubstituted C₂₋₆ alkenyl, substituted or unsubstituted C₂₋₆ alkynyl, substituted or unsubstituted C₁₋₆ heteroalkyl, substituted or unsubstituted C₃₋₆ cycloalkyl, substituted or unsubstituted C₃₋₆ heterocycloalkyl, substituted or unsubstituted aryl, substituted or unsubstituted heteroaryl, or mixtures thereof.

In still other examples, diaryliodonium salts of Formula VI can be used.

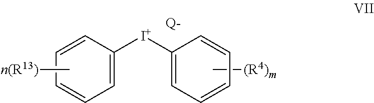

wherein Q is a counterion such as F, Cl, Br, NO₂, HSO₄, HCO₃, OTs, OTf, and CH₃CO₂; m is 0, 1, 2, 3, 4, or 5; n is 0, 1, 2, 3, 4, or 5; R⁴ is as defined above; and R¹³ is H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups.

In specific examples, R⁴ is F, Cl, Br, I, OH, SH, C(O)H, CO₂H, C(O)R³, CO₂R³, OR³, NO₂, CN, N(R³)₂, NC(O)R³, C(O)N(R³)₂, CNR³, Si(R³)₃, SO₂R³, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups; and m is 1. In other examples, m is 2 or 3. In other examples, n is 2 or 3. In still other examples, R⁴ is F, Cl, Br, I, OH, SH, C(O)H, CO₂H, C(O)R⁵, CO₂R⁵, OR⁵, NO₂, CN, N(R⁵)₂, NC(O)R⁵, C(O)N(R⁵)₂, CNR⁵, Si(R⁵)₃, SO₂R⁵, substituted or unsubstituted C₁₋₂₀ alkyl, or substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups. In still other examples, n is 3 and each R¹³ is methyl. In still other examples, each R⁴ is substituted or unsubstituted C₁₋₆ alkyl, substituted or unsubstituted C₂₋₆ alkenyl, substituted or unsubstituted C₂₋₆ alkynyl, substituted or unsubstituted C₁₋₆ heteroalkyl, substituted or unsubstituted C₃₋₆ cycloalkyl, substituted or unsubstituted C₃₋₆ heterocycloalkyl, substituted or unsubstituted aryl, substituted or unsubstituted heteroaryl, or mixtures thereof.

Stoichiometric amounts of the carbonyl compound and the aryl or heteroaryl halide are used in the disclosed methods.

### Pd Catalysts

The disclosed methods are conducted in the presence of a catalytic amount of palladium and/or at least one palladium compound (e.g., a chelate or complex of palladium) in which the palladium has a valence of zero, 1 or 2. In most examples, however, best results are obtain when the palladium or palladium compound has a valance of 2, i.e., Pd(II).

To start up the reaction for continuous operation, or in conducting the first of a series of batch reactions, it is often desirable to use fresh catalyst, the term “fresh” being used herein to refer to unused catalyst, and to thereby distinguish from recycled catalyst residues. The term does not refer to the time at which the catalyst was formulated, as fresh catalyst can be preformed and stored under suitable conditions prior to use. After reaction initiation and commencement of catalyst recycle in a continuous operation, recycled catalyst residues can be charged continuously or portion-wise to the reactor and implemented whenever deemed necessary or desirable by feed of fresh catalyst. Similarly, when conducting the reaction as a series of batch operations, the second and subsequent reactions can utilize recycled catalyst residues either as the sole catalyst or in combination with fresh catalyst.

While palladium metal or various types of palladium compounds can be used in forming fresh catalyst for the reaction, the use of salts of palladium is preferable because fresh catalyst compositions formed from palladium salts appear to have greater activity at least as compared to those made from palladium metal itself. Of the salts, palladium(II) salts such as the Pd(II) halides (e.g., palladium(II)chloride, palladium(II)bromide, palladium(II)iodide), Pd(II) carboxylates (e.g., palladium(II)acetate, palladium(II)propionate, palladium(II)trifluoroacetate), and other palladium(II) salts (e.g., palladium(II)trifluoromethanesulfonate, palladium(II)tetrafluoroborate, palladium(II)hexafluorophosphate, palladium(II)hexafluoroantimonate) can be used. As disclosed herein, palladium(II)trifluoroacetate or palladium(II)acetate are preferred in many examples of the disclosed methods. Palladium(0) salts can also be used as catalysts, such as Palladium(0)tetrakistriphenylphosphine and palladium(0)-bis(dibenzylideneacetone).

### Ligand

The disclosed methods are conducted in the presence of at least one ligand. In specific examples, the ligand is an organophosphine having the formula R⁶R⁷R⁸P, where R⁶, R⁷, and R⁸ are each, independent of one another, substituted or unsubstituted C_{2-20 l -}2o alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups. In further examples, the organophosphine can have the formula R⁶R⁷R⁸P, where R⁶, R⁷, and R⁸ are each, independent of one another, substituted or unsubstituted C₁₋₁₀ alkyl, or substituted or unsubstituted C₁₋₈ cycloalkyl, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups. In more specific examples, the organophosphine ligand can be triisopropyl phosphine, trimethyl phosphine, tricyclohexyl phosphine, or mixtures thereof.

In other examples, the ligand is an organophosphine having the formula R⁹R¹⁰R¹¹P, where R⁵ and R⁶ are each, independent of one another, substituted or unsubstituted aryl or substituted or unsubstituted heteroaryl, and R7 is a substituted or unsubstituted C₁₋₈ cycloalkyl. In specific examples, R⁵ and R⁶ can be each, independent of one another, substituted or unsubstituted phenyl or naphtyl.

In some examples, the organophoshine is neomenthyl-diphenylphosphine, trifurylphosphine, tri(o-tolyl)phosphine, tri(cyclohexyl)phosphine, tri(t-butyl)phosphine, dicyclohexylphenylphosphine, 1,1′-bis(di-t-butylphosphino)ferrocene, 2-dicyclohexylphosphino-2′-dimethylamino-1,1′-biphenyl, 2-(di-t-butylphosphino)biphenyl, 2-(dicyclohexylphosphino)biphenyl, 2,2′-bis(diphenylphosphino)-1,1′-binaphthyl, xantphos, and tri(tert-butyl)phosphine. Alternative examples also include 2-dicyclohexylphosphino-2′,6′-dimethoxybiphenyl, 2-dicyclohexyl-2′,4′,6′-triisopropylbiphenyl, and 1,2,3,4,5-pentamethyl-1′-(di-t-butylphosphino)ferrocene). 2-(di-tert-butylphosphino)biphenyl, 2-(dicyclohexylphosphino)biphenyl, 2,2′-bis (diphenylphosphino)-1,1′-binaphthyl, xantphos, tri(tert-butyl)phosphine, and the like. In some examples, DMSO type ligand and pyridine (bipyridine) can be used.

In still other examples, the ligand can be a sulfonate, sulfinimide, sulfone, sulfoxide or sulfide containing ligand. In specific examples, the ligand can be a bis-sulfinimide ligand. In another example, the ligand is a dipyridin-5-one. These ligands are particularly useful when using the diaryliodonium salts as the aryl source. Examples of such ligands are below.

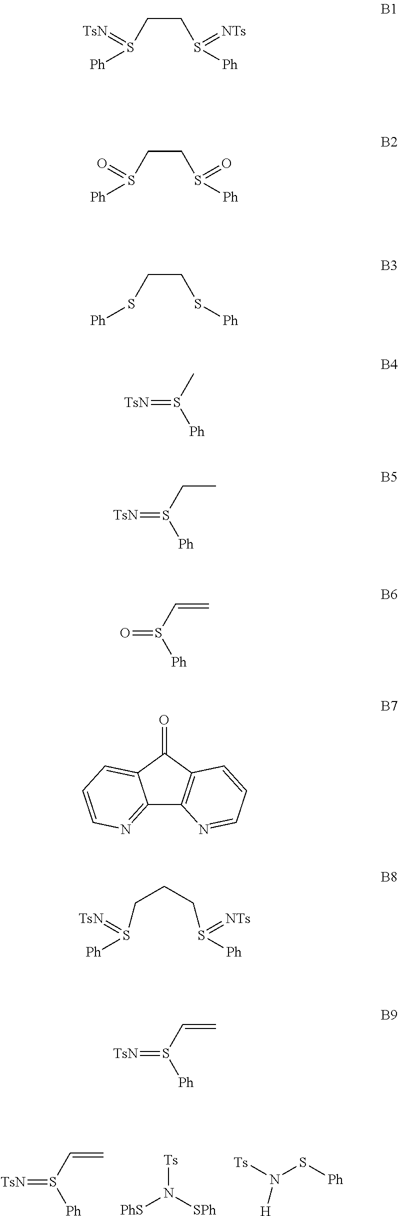

In a preferred aspect, the ligand is B1.

The amount of ligand can vary depending on the particular carbonyl compound. In certain examples, from about 0.1 to about 1 equivalents of the ligand can be used per equivalent of the carbonyl compound. For example, from about 0.1 to about 0.9 equivalents, from about 0.1 to about 0.8 equivalents, from about 0.1 to about 0.7 equivalents, from about 0.1 to about 0.6 equivalents, from about 0.1 to about0.5 equivalents, from about 0.1 to about 0.4 equivalents, from about 0.1 to about 0.3 equivalents, from about 0.1 to about 0.2 equivalents, from about 0.2 to about 1 equivalents, from about 0.2 to about 0.9 equivalents, from about 0.2 to about 0.8 equivalents, from about 0.2 to about 0.7 equivalents, from about 0.2 to about 0.6 equivalents, from about 0.2 to about 0.5 equivalents, from about 0.2 to about 0.4 equivalents, from about 0.2 to about 0.3 equivalents, from about 0.3 to about 1 equivalents, from about 0.3 to about 0.9 equivalents, from about 0.3 to about 0.7 equivalents, from about 0.3 to about 0.6 equivalents, from about 0.3 to about 0.5 equivalents, from about 0.3 to about 0.4 equivalents, from about 0.4 to about 1 equivalents, from about 0.4 to about 0.9 equivalents, from about 0.4 to about 0.8 equivalents, from about 0.4 to about 0.7 equivalents, from about 0.4 to about 0.6 equivalents, from about 0.4 to about 0.5 equivalents, from about 0.5 to about 1 equivalents, from about 0.5 to about 0.9 equivalents, from about 0.5 to about 0.8 equivalents, from about 0.5 to about 0.7 equivalents, from about 0.5 to about 0.6 equivalents, from about 0.6 to about 1 equivalents, from about 0.6 to about 0.9 equivalents, from about 0.6 to about 0.8 equivalents, from about 0.6 to about 0.7 equivalents, from about 0.7 to about 1 equivalents, from about 0.7 to about 0.9 equivalents, from about 0.7 to about 0.8 equivalents, from about 0.8 to about 1 equivalents, from about 0.8 to about 0.9 equivalents, or from about 0.9 to about 1 equivalents of ligand can be used per equivalent of the carbonyl compound.

### Promoter

The disclosed methods can also be conducted in the presence of a promoter. The promoter can be a silver, copper, potassium, sodium, lithium, magnesium or cesium compound (e.g., salt or chelate or complex). A suitable promoter is a compound that can form strong bonds with the X group on the aryl or heteroaryl halide/tosylate/triflate.

Silver compounds such as silver carbonate, silver sulfate, silver nitrate, silver formate, silver acetate, silver proprionate, silver nitrate, silver chloride, silver bromide, silver iodide, silver trifluoromethanesulfonate, silver tetrafluoroborate, silver hexafluorophosphate, silver hexafluoroantimonate, or any mixture thereof can be used as the promoter. In many examples herein, the promoter can be silver trifluoroacetate.

Copper compounds such as copper acetylacetonates, copper alkylaceto-acetates, or other forms of copper can be used as the promoter. Especially suitable copper compounds for use herein are salts, e.g., divalent copper salts such as copper(II)chloride, copper(II)bromide, copper(II)iodide, or copper(II) carboxylates, like copper(II)acetate, and copper(II)propionate. In certain examples, the promoter is copper(II)trifluoroacetate.

Potassium compounds can also be used as promoters. For example, potassium chloride, potassium bromide, potassium iodide, potassium acetate, potassium trifluoroacetate, potassium trifluoromethanesulfonate, potassium tetrafluoroborate, potassium hexafluorophosphate, potassium hexafluoroantimonate, or any mixture thereof can be used. Potassium promoters are especially useful in that they are less expensive than the heavy metal based promoters. These promoters can be used with diaryliodonium substrates and/or the bis-sulfonamide ligands, as disclosed herein.

Sodium compounds can also be used as promoters. For example, sodium chloride, sodium bromide, sodium iodide, sodium acetate, sodium trifluoroacetate, sodium trifluoromethanesulfonate, sodium tetrafluoroborate, sodium hexafluorophosphate, sodium hexafluoroantimonate, or any mixture thereof can be used.

Lithium compounds can also be used as promoters. For example, lithium chloride, lithium bromide, lithium iodide, lithium acetate, lithium trifluoroacetate, lithium trifluoromethanesulfonate, lithium tetrafluoroborate, lithium hexafluorophosphate, lithium hexafluoroantimonate, or any mixture thereof can be used.

Cesium compounds can also be used as promoters. For example, cesium chloride, cesium bromide, cesium iodide, cesium acetate, cesium trifluoroacetate, cesium trifluoromethanesulfonate, cesium tetrafluoroborate, cesium hexafluorophosphate, cesium hexafluoroantimonate, or any mixture thereof can be used.

The amount of promoter can vary depending on the particular carbonyl compound. In certain examples, from about 0.2 to about 5 equivalents of the promoter can be used per equivalent of the carbonyl compound. For example, from about 0.2 to about 4.5 equivalents, from about 0.2 to about 4 equivalents, from about 0.2 to about 3.5 equivalents, from about 0.2 to about 3 equivalents, from about 0.2 to about 2.5 equivalents, from about 0.2 to about 2 equivalents, from about 0.2 to about 1.5 equivalents, from about 0.2 to about 1 equivalents, from about 0.2 to about 0.5 equivalents, from about 0.5 to about 4.5 equivalents, from about 0.5 to about 4 equivalents, from about 0.5 to about 3.5 equivalents, from about 0.5 to about 3 equivalents, from about 0.5 to about 2.5 equivalents, from about 0.5 to about 2 equivalents, from about 0.5 to about 1.5 equivalents, from about 0.5 to about 1 equivalents, from about 1 to about 5 equivalents, from about 1 to about 4.5 equivalents, from about 1 to about 4 equivalents, from about 1 to about 3.5 equivalents, from about 1 to about 3 equivalents, from about 1 to about 2.5 equivalents, from about 1 to about 2 equivalents, from about 1 to about 1.5 equivalents, from about 1.5 to about 5 equivalents, from about 1.5 to about 4.5 equivalents, from about 1.5 to about 4 equivalents, from about 1.5 to about 3.5 equivalents, from about 1.5 to about 3 equivalents, from about 1.5 to about 2.5 equivalents, from about 1.5 to about 2 equivalents, from about 2 to about 5 equivalents, from about 2 to about 4.5 equivalents, from about 2 to about 4 equivalents, from about 2 to about 3.5 equivalents, from about 2 to about 3 equivalents, from about 2 to about 2.5 equivalents, from about 2.5 to about 5 equivalents, from about 2.5 to about 4.5 equivalents, from about 2.5 to about 4 equivalents, from about 2.5 to about 3.5 equivalents, from about 2.5 to about 3 equivalents, from about 3 to about 5 equivalents, from about 3 to about 4.5 equivalents, from about 3 to about 4 equivalents, from about 3 to about 3.5 equivalents, from about 3.5 to about 5 equivalents, from about 3.5 to about 4.5 equivalents, from about 3.5 to about 4.0 equivalents, from about 4 to about 5 equivalents, from about 4 to about 4.5 equivalents, or from about 4.5 to about 5 equivalents of the promoter can be used per equivalent of the carbonyl compound.

### Solvent

In the disclosed methods, solvents are not always required, though in most case they can be used. Solvents which can be used include one or more of the following: ketones, for example, acetone, methyl ethyl ketone, diethyl ketone, methyl n-propyl ketone, acetophenone, and cyclohexanone; linear, poly and cyclic ethers, for example, diethyl ether, di-n-propyl ether, di-n-butyl ether, ethyl n-propyl ether, glyme (the dimethyl ether of ethylene glycol), diglyme (the dimethyl ether of diethylene glycol), tetrahydrofuran, 1,4-dioxane, 1,3-dioxolane, and similar compounds; and aromatic hydrocarbons, for example, toluene, ethyl benzene, xylenes, and similar compounds. Alcohols, for example, methanol, ethanol, 1-propanol, 2-propanol, isomers of butanol, isomers of pentanol, halogenated alcohols such as trifluormethanol, pentafluoroethanol, or hexafluoroisopropanol can be used as solvents alone or in combination with ketone or ether solvents. Further examples of suitable solvents include dimethylformamide, DME dimethoxyethane, dimethylacetamide,

N-methylpyrrolidone, and dimethylsulfoxide. In most reactions, however, the solvent can be an ether, such as 1,4-dioxane with one or more such more alcohols, such as hexafluoroisopropanol.

If used in the disclosed methods, the amount of solvent can vary depending on the particular carbonyl compound. In certain examples, from about 1 to about 200 equivalents of the solvent can be used per equivalent of the carbonyl compound. For example, from about 1 to about 150 equivalents, from about 1 to about 100 equivalents, from about 1 to about 50 equivalents, from about 1 to about 10 equivalents, from about 10 to about 200 equivalents, from about 10 to about 150 equivalents, from about 10 to about 100 equivalents, from about 10 to about 50 equivalents, from about 50 to about 200 equivalents, from about 50 to about 150 equivalents, from about 50 to about 150 equivalents, from about 50 to about 100 equivalents, from about 100 to about 200 equivalents, from about 100 to about 150 equivalents, or from about 150 to about 200 equivalents, of the solvent can be used per equivalent of the carbonyl compound.

### Conditions

In the disclosed methods, the carbonyl compound, aryl or heteroaryl halide/tosylate/triflate, palladium catalyst, ligand, and promoter be combined in any order. For example, the palladium catalyst can be combined with the ligand and promoter, and then this mixture can be added to the aryl or heteroaryl halide/tosylate/triflate and carbonyl compound. Alternatively, the carbonyl compound can be combined with the palladium catalyst, followed by the addition of the ligand, promoter and aryl or heteroaryl halide/tosylate/triflate. In another alternative, the aryl or heteroaryl halide/tosylate/triflate and carbonyl compound are combined, followed by addition of the ligand, promoter, and then palladium compound. Still further, the carbonyl compound, aryl or heteroaryl halide/tosylate/triflate, palladium catalyst, ligand, and promoter are combined. The combination of these materials can be accomplished by methods known in the art. Typically, the addition can be accompanied by mixing, stirring, shaking or other form of agitation. Typically the materials are combined under an inert atmosphere, such as under N₂ or argon.

Further, the combination of these materials can be conducted at elevated temperature, e.g., from about 30° C. to about 225° C., from about 50° C. to about 200° C., from about 100° C. to about 150° C., from about 100° C. to about 225° C., from about 150° C. to about 225° C., from about 30° C. to about 100° C., from about 50° C. to about 100° C., from about 30° C. to about 50° C., or from about 75° C. to about 200° C. In certain examples, the materials can be combined at room temperature.

The resulting product of the disclosed methods is β-arylation of the carbonyl compound. Thus, referencing the carbonyl compound of Formula I and II above, and the aryl or heteroaryl halide/tosylate/triflate of Formula IV or the diaryliodinium salts of Formula VI above, the product can be represented by Formula I-P and II-P.

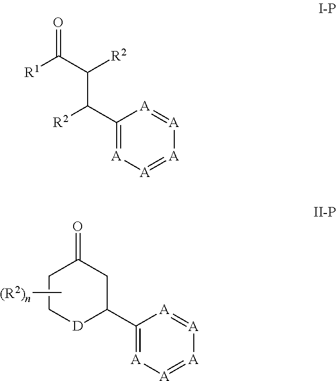

where variables R¹, R², n, A, and D are as previously defined. It is notable that the disclosed methods are selective in that the arylation occurs at the β-carbon. Thus, the amount of arylation at the α-carbon is much less, e.g., less than 10 wt. %, of compounds of Formula I-P or II-P where arylation occurred at the β-carbon.

### EXAMPLES

The following examples are set forth below to illustrate the methods, compositions, and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### General Procedure for the β-Arylation of Ketones

An 8 mL vial was charged with Pd(TFA)₂ (10 mol %), AgTFA (2.0 equiv.), hexafluoroisopropanol (1 mL), ketone (2.5 equiv.) and aryl or heteroaryl iodide/bromide/tosylate/triflate (0.4 mmol). The vial was sealed with a PTFE lined cap and transferred to a glove box. The vial was opened and 1,4-dioxane (1 mL) and P(i-Pr)₃ (20 mol %) were added under N₂ purging. The vial was then sealed again and heated in a pie-block at 80° C. for 12 hours under stirring. After cooled to room temperature, the mixture was filtered through a small plug of silica gel and eluted with diethyl ether. The solvent was then removed under vacuum and flash column chromatography (hexane/ethyl acetate or DCM/methanol) of the residue gave the β-arylation product.

### Example 1

To demonstrate the feasibility of the disclosed methods, cyclohexanone (1a) and iodobenzene (2a) were used as the model substrates. A variety of palladium pre-catalysts, ligands, solvents and additives were examined (Table 1). Ultimately, It was discovered that use of palladium trifluoroacetate/triisopropyl phosphine as the pre-catalyst/ligand and silver trifluoroacetate as the promoter in 1,4-dioxane/hexafluoroisopropanol (HFIP) as mixed-solvents afforded the desired β-arylation product (3-phenyl cyclohexanone, 3a) in a 76% yield, along with 10% of biphenyl formed through the dimerization of iodobenzene (Eq. 1). Surprisingly, neither over-oxidation to 3-phenyl-cyclohexenone nor the α-arylation product was observed. The combination of electron-rich phosphine-Pd complexes and aryl or heteroaryl halides has been extensively employed in the ketone α-arylation reactions; however, while a similar combination is employed in the disclosed methods, this reaction proceeded with complete site-selectivity for the β-position. While not wishing to be bound by theory, this selectivity is believed to be explained by the fact that the Buchwald-Hartwig-Miura arylation typically uses stoichiometric bases to generate the corresponding enolates, but the disclosed methods operate under acidic conditions, which triggered a different activation mode (FIG. 1c).

TABLE 1
Examples of β-Arylation of Cyclohexanone with Iodobenzene

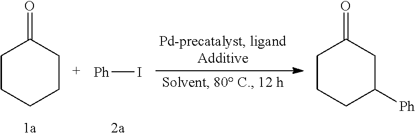 [SMILES: O=C1CCCCC1 ; Ic1ccccc1 ; O=C1CCCC(c2ccccc2)C1]

| | | Catalyst | | Additive | | Yield |
|---|---|---|---|---|---|---|
| Entry | 1a:2a | (mol %) | Ligand (mol %) | (mol %) | Solvent | (%) |
| 1 | 1:4 | Pd(TFA)₂ (10) | DMSO (20) | AgOAc (150) | 1,4-dioxane/TFA 1:1 | 42 |
| 2 | 1:4 | Pd(TFA)₂ (10) | DMSO (20) | AgTFA (150) | 1,4-dioxane/TFA 1:1 | 46 |
| 3 | 1:4 | Pd(TFA)₂ (10) | DMSO (20) | AgTFA (150) | 1,4-dioxane/HFIP | 24 |
| | | | | | 1:1 | |
| 4 | 1:4 | Pd(TFA)₂ (10) | — | AgTFA (150) | 1,4-dioxane/HFIP | 12 |
| | | | | | 1:1 | |
| 5 | 1:4 | Pd(TFA)₂ (10) | 2,2′-bipyridine | AgTFA (150) | 1,4-dioxane/HFIP | 19 |
| | | | (10) | | 1:1 | |
| 6 | 1:4 | Pd(TFA)₂ (10) | L1 (10) | AgTFA (150) | 1,4-dioxane/HFIP | 11 |
| | | | | | 1:1 | |
| 7 | 1:4 | Pd(TFA)₂ (10) | L2 (10) | AgTFA (150) | 1,4-dioxane/HFIP | 27 |
| | | | | | 1:1 | |
| 8 | 1:4 | Pd(TFA)₂ (10) | pyridine (20) | AgTFA (150) | 1,4-dioxane/HFIP | trace |
| | | | | | 1:1 | |
| 9 | 1:4 | Pd(TFA)₂ (10) | L3 (20) | AgTFA (150) | 1,4-dioxane/HFIP | trace |
| | | | | | 1:1 | |
| 10 | 1:4 | Pd(TFA)₂ (10) | L4 (15) | AgTFA (150) | 1,4-dioxane/HFIP | 4 |
| | | | | | 1:1 | |
| 11 | 1:4 | Pd(TFA)₂ (10) | L5 (10) | AgTFA (150) | 1,4-dioxane/HFIP | trace |
| | | | | | 1:1 | |
| 12 | 1:4 | Pd(TFA)₂ (10) | L6 (15) | AgTFA (150) | 1,4-dioxane/HFIP | 2 |
| | | | | | 1:1 | |
| 13 | 1:4 | Pd(TFA)₂ (10) | dppm (15) | AgTFA (150) | 1,4-dioxane/HFIP | 26 |
| | | | | | 1:1 | |
| 14 | 1:4 | Pd(TFA)₂ (10) | dppe (15) | AgTFA (150) | 1,4-dioxane/HFIP | trace |
| | | | | | 1:1 | |
| 15 | 1:4 | Pd(TFA)₂ (10) | dppb (15) | AgTFA (150) | 1,4-dioxane/HFIP | trace |
| | | | | | 1:1 | |
| 16 | 1:4 | Pd(TFA)₂ (10) | dppf (15) | AgTFA (150) | 1,4-dioxane/HFIP | trace |
| | | | | | 1:1 | |
| 17 | 1:4 | Pd(TFA)₂ (10) | PPh₃ (20) | AgTFA (200) | 1,4-dioxane/HFIP | 39 |
| | | | | | 1:1 | |
| 18 | 1:4 | Pd(PPh₃)₄ (10) | — | AgTFA (200) | 1,4-dioxane/HFIP | 21 |
| | | | | | 1:1 | |
| 19 | 1:4 | Pd(TFA)₂ (10) | P(p-tol)₃ (20) | AgTFA (200) | 1,4-dioxane/HFIP | 27 |
| | | | | | 1:1 | |
| 20 | 1:4 | Pd(TFA)₂ (10) | P(o-tol)₃ (20) | AgTFA (200) | 1,4-dioxane/HFIP | 5 |
| | | | | | 1:1 | |
| 21 | 1:4 | Pd(TFA)₂ (10) | PCy₃ (20) | AgTFA (200) | 1,4-dioxane/HFIP | 48 |
| | | | | | 1:1 | |
| 22 | 1:4 | Pd(TFA)₂ (10) | P(i-Pr)₃ (20) | AgTFA (200) | 1,4-dioxane/HFIP | 51 |
| | | | | | 1:1 | |
| 23 | 1:1 | Pd(TFA)₂ (10) | P(i-Pr)₃ (20) | AgTFA (200) | 1,4-dioxane/HFIP | 67 |
| | | | | | 1:1 | |
| 24 | 2.5:1 | Pd(TFA)₂ (10) | P(i-Pr)₃ (20) | AgTFA (200) | 1,4-dioxane/HFIP | 76 |
| | | | | | 1:1 | |

L1 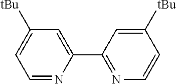 [SMILES: CC(C)(C)c1ccnc(-c2cc(C(C)(C)C)ccn2)c1]
L2 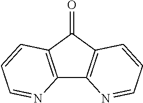 [SMILES: O=C1c2cccnc2-c2ncccc21]
L3 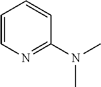
L4 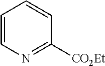 [SMILES: CCOC(=O)c1ccccn1]
L5 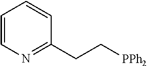
L6

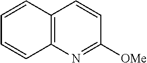 [SMILES: COc1ccc2ccccc2n1]

### Example 2

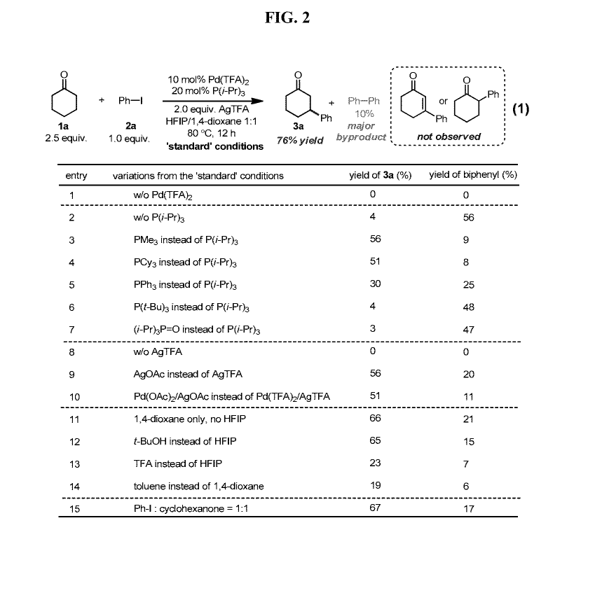

A set of control experiments were conducted to further demonstrate the disclosed methods (FIG. 2). In the absence of palladium, no product was obtained, suggesting the pivotal position of palladium in the catalytic cycle (entry 1). Electron-rich ligands were seldom used in the Pd-catalyzed dehydrogenation reactions (Campbell, et al., Overcoming the “oxidant problem”: strategies to use O₂ as the oxidant in organometallic C—H oxidation reactions catalyzed by Pd (and Cu). Acc. Chem. Res. 45, 851-863 (2012); Hong, et al., Pd-Catalyzed Semmler-Wolff reactions for the conversion of substituted cyclohexenone oximes to primary anilines. J. Am. Chem. Soc. Articles ASAP, 29 Aug. 2013 (DOI: 10.1021/ja4073172)). However, for the disclosed β-arylation reaction, electron-rich phosphines play a role in inhibiting the aryl or heteroaryl dimerization and promoting formation of the desired product, though the exact reason is unclear (entries 2-7). In the absence of triisopropyl phosphine or use of triisopropyl phosphine oxide, biphenyl was observed as the major product with only a trace amount of the β-arylated ketone formed (entries 2 and 7). Other electron-rich phosphines, such as trimethyl phosphine and tricyclohexyl phosphine, also proved to be efficient (entries 3 and 4). In contrast, use of less electron-rich triphenyl phosphine or sterically-hindered tri-tert-butyl phosphine led to formation of a considerable amount of biphenyl (entries 5 and 6). Nitrogen-based ligands were found much less effective (for details, see Table 1). Silver salts were utilized to facilitate iodide-carboxylate exchange, which is expected to be crucial for regenerating the active Pd(II) catalyst (entry 8). Replacing the trifluoroacetate counterion of the silver salts with acetate resulted in decreased yields (entries 9 and 10). Solvent effects were also surveyed (entries 11-14): while 1,4-dioxane is most suitable for this transformation, addition of mildly acidic HFIP (pKₐ 9.3 in H₂O) is beneficial likely because it accelerates the protonation of Pd-enolates to regenerate the Pd(II) catalysts (Step F, FIG. 1c), which in turn should diminish side reactions, i.e. over-oxidations. On the other hand, HFIP is not acidic enough to protonate phosphine ligands, whereas using stronger acids, such as trifluoroacetic acid (TFA), as a co-solvent was found detrimental. Finally, when cyclohexanone and iodobenzene were added in an equimolar ratio, the desired 3-phenyl cyclohexanone was still provided in 67% yield (entry 15).

### Example 3

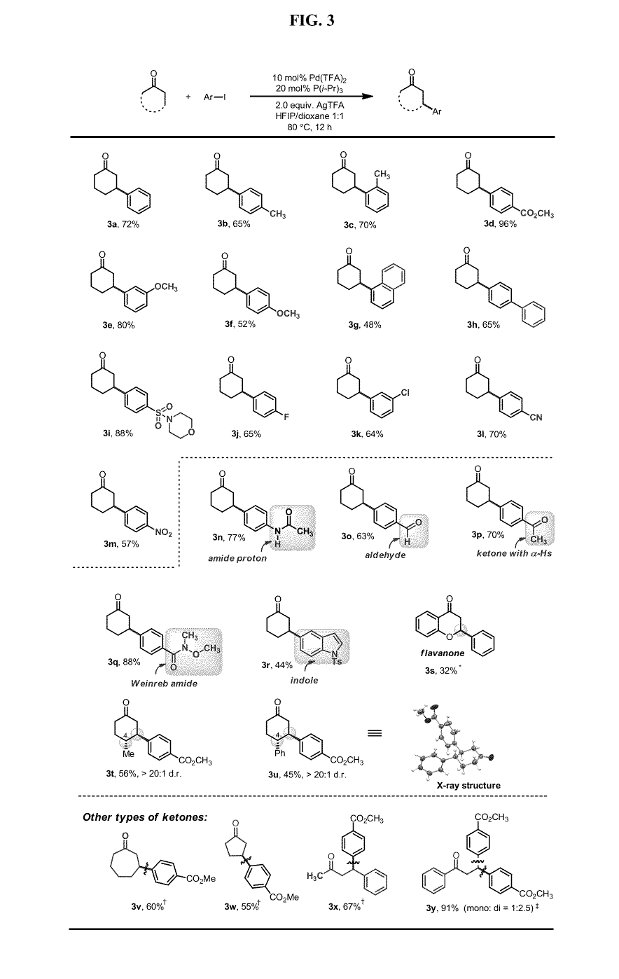

The substrate diversity was then investigated (FIG. 3). Aryl iodides containing arenes with different electronic properties (electron-rich and -poor) all participated to give the corresponding β-arylated ketones (3a-3m). In addition, substitutions on the aryl or heteroaryl group at the ortho-, meta- or para positions are all tolerated. Furthermore, a broad range of functional groups, including aryl ethers, cyanides, aryl chlorides, fluorides, naphthalene, protected indoles, carboxylic esters, nitro group, and sulfonamides, are compatible under the reaction conditions. These features indicate that this Pd-catalyzed direct β-arylation exhibits comparable reactivity and substrate diversity as the aryl or heteroaryl-conjugate addition reactions but using fewer steps or redox operations. It is also surprising that products that are more difficult to prepare via conventional aryl or heteroaryl-metal-based 1,4-additions, such as arenes that contain base- or nucleophile-sensitive groups (e.g., amide protons 3n, ketones with enolizable hydrogens 3p, aldehydes 3o, Weinreb amide 3q), can also be arylated at the β-carbon using the disclosed methods. The high chemoselectivity is likely due to the mild reaction conditions (base-free) as well as the absence of stoichiometric aryl or heteroaryl-metal nucleophiles.

### Example 4

The diversity of the ketone component was also examined The β-arylation of ketones bearing a stereocenter at the C4 position proceeded with excellent diastereoselectivity (>20:1) to give the trans products (3t and 3u). Besides the 6-membered ring ketones, cyclopentanone and cycloheptanone can also participate in the reaction (3v, 3w); acyclic ketones also proved to be suitable substrates (3x, 3y). The broad substrate diversity demonstrated here is clearly complementary to the previous directing group and photo-redox β-arylation methods. Interestingly, when propiophenone was employed as the substrate, a mixture of mono- and di-arylated products was isolated in 91% yield with a 1:2.5 ratio. The tendency of propiophenone to give diarylation products likely comes from its flexible conformation permitting free bond rotation, which in turn, results in a second ketone dehydrogenation via β-H elimination.

### Example 5

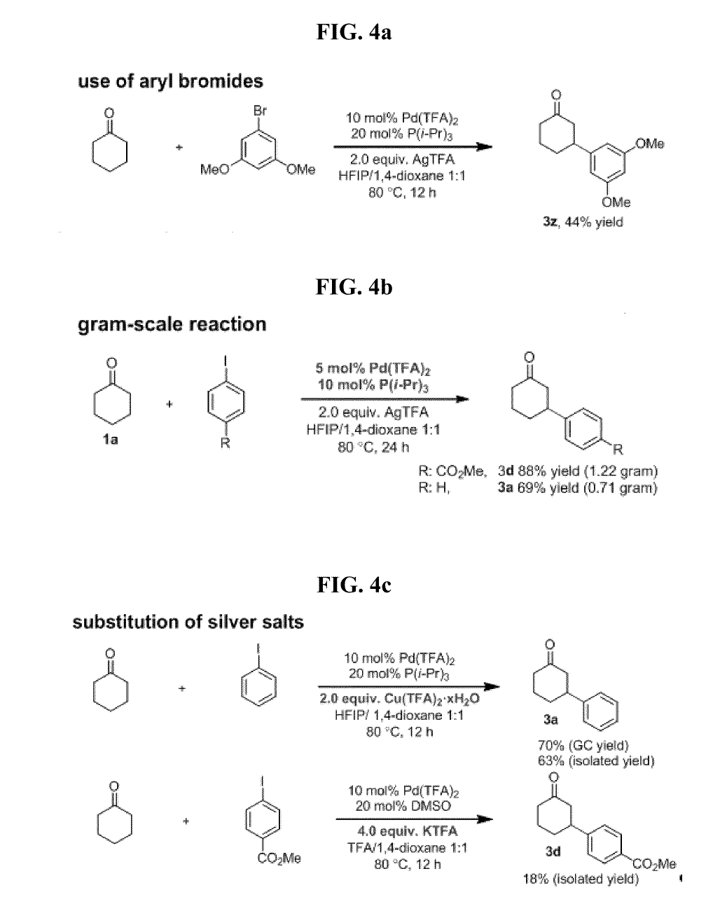

It is known that aryl bromides are less reactive than aryl iodides in the palladium-mediated oxidative additions; nevertheless, it was found that aryl bromides are suitable substrates in the disclosed methods (FIG. 4a). In addition, the disclosed Pd-catalyzed β-arylation proved to be readily scalable; on a gram scale using cyclohexanone and methyl 4-iodobenzoate as reactants, the β-arylation product was isolated in 88% yield with a lower catalyst loading (FIG. 4b). Efforts have also been set forth to examine whether the silver promoter can be substituted with more economically viable reagents. It was found that replacement of silver trifluoroacetate with copper(II) trifluoroacetate only slightly reduced the yield (FIG. 4c). In addition, the use of potassium trifluoroacetate as the promoter was also found to deliver the β-arylation product. Although the catalyst turnover is low (ca 2) at this early stage, it shows the potential to develop “stoichiometric heavy metal-free” conditions for this transformation (Zhao, et al., Palladium-catalyzed alkylation of ortho-C(sp²)—H bonds of benzylamide substrates with alkyl halides. Org. Lett. 13, 4850 (2011)).

### Example 6

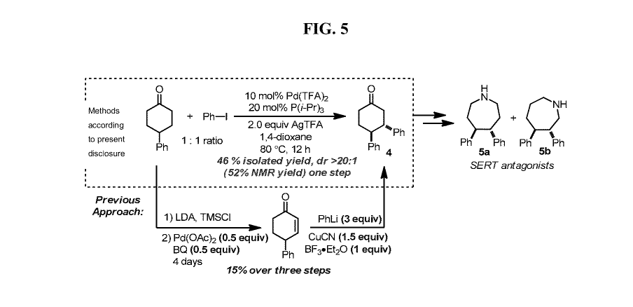

Application of the disclosed methods to synthesize a key intermediate, trans-3,4-diphenyl-cyclohexan-1-one (4), for SERT antagonist 5a/b is demonstrated (FIG. 5). The previous approach, using classical dehydrogenation and 1,4-addition (with super-stoichiometric reagents), required three steps and provided a 15% overall yield. By employing the disclosed β-arylation method, the desired product (4) was obtained in one single step with a triple-increased yield from same ketone material and just one equiv of PhI. In addition, given its high chemoselectivity and broad substrate, this direct β-arylation approach can potentially provide rapid access to various new analogues for the SERT-antagonist-drug development (e.g., compound 3t, FIG. 3).

### Example 7

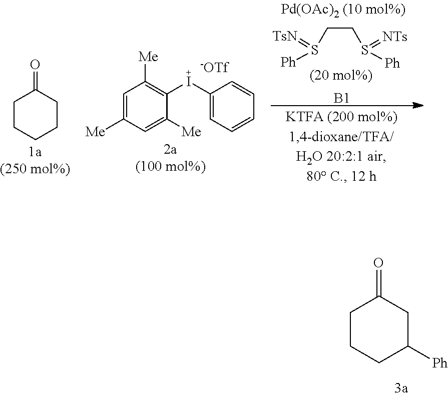

In a general procedure for synthesizing palladium-catalyzed β-arylation of simple ketones with mesitylaryliodonium salts, an 8 mL vial was charged with Pd(OAc)₂ (9.0 mg, 0.1 equiv.), B1 (24 mg, 0.1 equiv.), KTFA (122 mg, 2.0 equiv.), 2a (189 mg, 0 4 mmol), cyclohexanone (98 mg, 2.5 equiv.), dioxane (2.0 mL), TFA (0.2 mL) and water (0.1 mL). The vial was sealed with a lined cap and heated in a pie-block at 80° C. for 12 hours under stirring. Then, the vial was allowed to cool to room temperature and the mixture was filtered through a small plug of silica gel, eluted with diethyl ether. The solvent was then removed in vacuo and flash column chromatography (hexane/ethyl acetate or DCM/methanol) of the residue gave the arylation product.

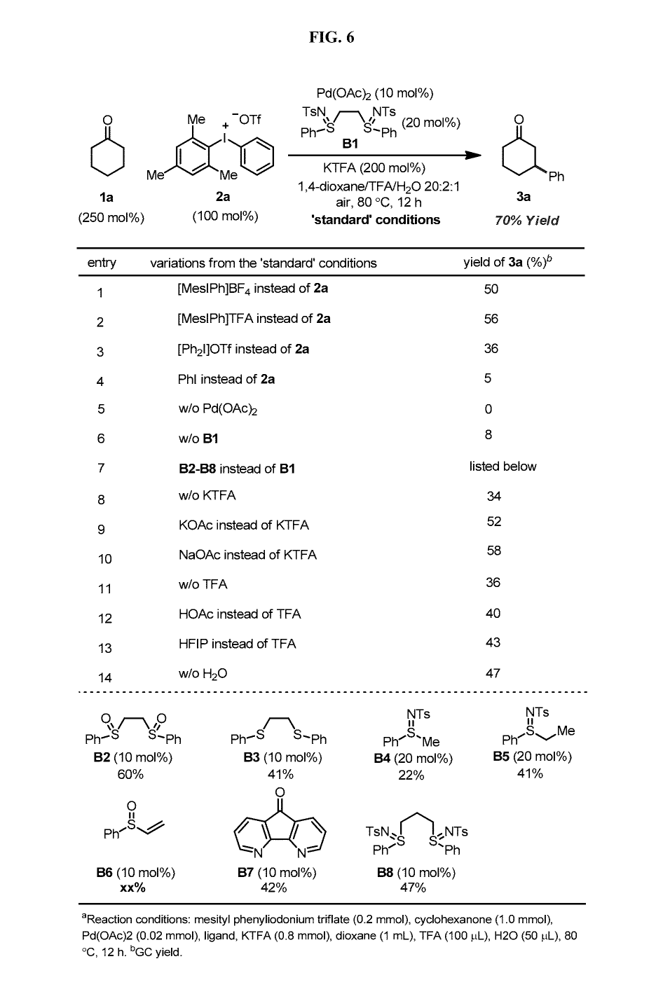

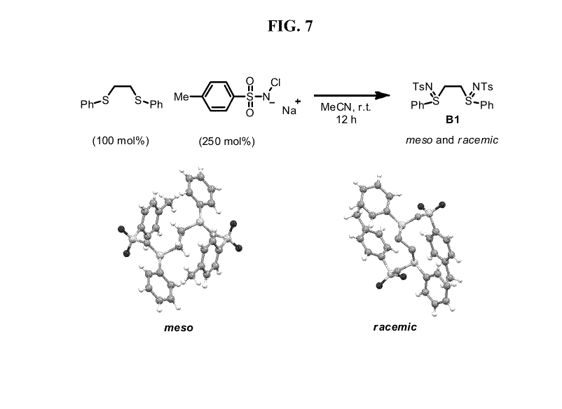

The reaction between cyclohexanone (1a) with mesitylphenyliodonium salt (2a) proceeds to give desired β-arylation product in a good yield under our optimized conditions (FIG. 6). Various mesitylphenyliodonium salts were suitable aryl sources for the reaction, giving the product in slightly reduced yields (entry 1-2). The inferior performance of diphenyliodonium salt is likely to be caused by the iodobenzene released during the reaction, which may deliver iodide ligand to Pd by oxidative addition and eventually suppress the reaction. On the other hand, the mesityliodide release by 2a during the reaction may be innocent by being too bulky to react with Pd(0). The reaction didn't give any product without either palladium or ligand. The structure of the bis-sulfinimide ligand (B1) is pivotal for the β-arylation reaction. B1 can be prepared from easily accessible bis-sulfide and Chlroamine T through a facile nitrene transfer reaction (Example 8 and FIG. 7). And ligands with racemic or meso conformation are able to give the β-arylation product in comparable yields. Corresponding sulfide (B3) and sulfoxide (B2) ligand with the same backbone gave lower yield under the same reaction conditions. Other π-acid ligands (B6 and B7) are also effective ligands for the reaction, although with lower efficiency. In addition, mono-dentate sulfinimide ligand (B4 and B5) and bis-sulfinimide ligand with an elongated backbone are not as good as B1. Another interesting aspect is the combination of a weak base KTFA and a strong acid TFA. Omission of either reagent will result in the great reduction of the yield (entry 8 and 11). It is believed that such a combination may act as a ‘buffer pair’ during the reaction. Acidic conditions are usually necessary for the palladium-mediated dehydrogenation of ketones. Thus the role of TFA is to maintain a low pH to facilitate the reaction, while, KTFA would react with the stoichiometric amount of HOTf generated during the reaction to prevent the acidity of the medium from being too low. Other inexpensive weak bases (entry 9-10) can also be used for the reaction while weaker acid gave lower yields (entry 12-13). Water also seems to be needed for the high efficiency of the reaction although the reason is unclear.

### Example 8

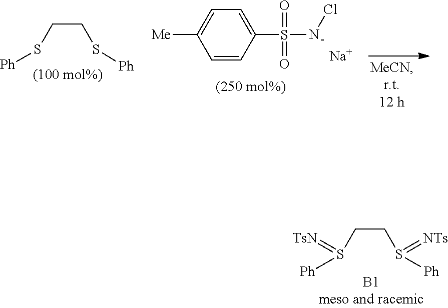

In a general procedure for the synthesis of B1, 1,2-bis(phenylthio)ethane (4.92 g, 20 mmol) and MeCN (80 mL) were added to a round bottom flask. To this mixture was added Chloramine T trihydrate (14.05 g, 2.5 equiv.). The mixture was stirred overnight at room temperature. The reaction was then quenched by adding 100 mL DCM. The precipitate was filtered off and mixture concentrated to give crude product. The crude product was dissolved in ca. 500 mL of acetone and insoluble solid filtered off. The filtrate was placed at room temperature. meso ligand would precipitate out in one day or two. After the meso ligand was collected by filtration, the filtrate was then again placed at room temperature for the precipitation of racemic ligand.

### Example 9

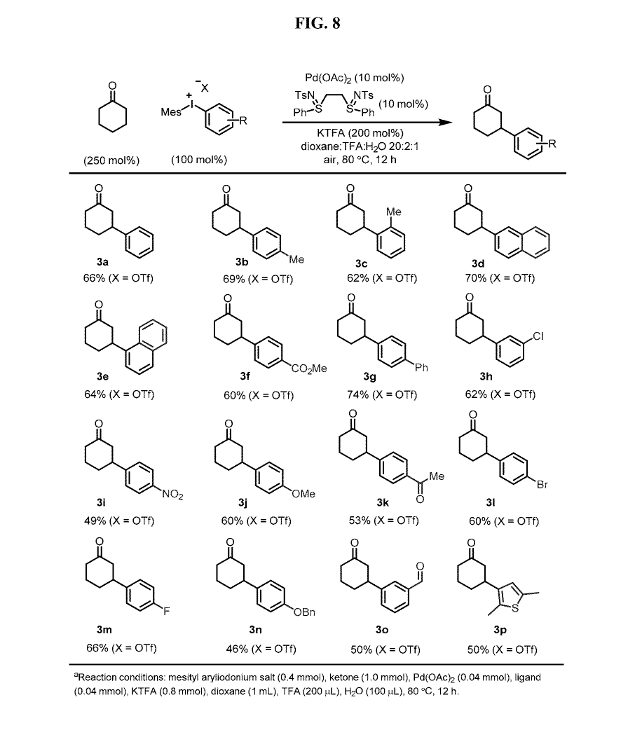

The substrate scope was then examined using the same conditions as in Example 7 (FIG. 8). Mesitylaryliodonium salts bearing arenes with a wide range of electronic properties (electron-rich and -poor) can participate in the reaction to give β-arylation products. Ortho, meta, and para substitution on the arenes are all tolerated under the reaction conditions. Product with the aryl bromide moiety, which is prone to undergo oxidative addition to Pd(0), can be accessed in a good yield. In addition, base- and nucleophile-sensitive functional groups, which are usually incompatible with the conditions of conjugate addition, can be tolerated under the reaction conditions (enolizable ketone 3k and aldehyde 3r). Heterocycles (e.g., 3p) are also compatible with the reaction conditions.

### Example 10

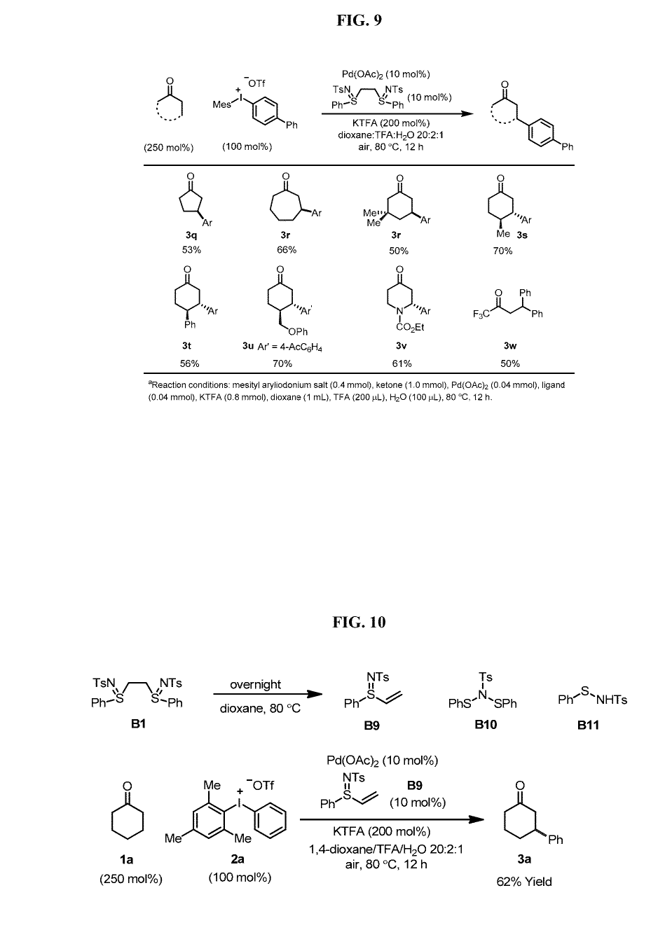

Cyclic ketones with different ring-size were tested under the same conditions of Example 7 (FIG. 9). Various cyclic ketones are compatible with the reaction condition (3q and 3r). Cyclohexanones with substitution on either 3- or 4-positions can participate in the reaction to give desired products with high diastereoselectivity (3r-u). In addition, N-protected 4-piperidone (3v) and some linear ketones are also suitable substrates for this β-arylation reaction.

### Example 11

NMR-Monitoring of the reaction mixture revealed that ligand B1 readily decomposed to several species (L9-L11) under the reaction (FIG. 10). It is believed that that B9 and B11 are formed through an E2 elimination of the ligand B1, though the pathway to form B10 is unclear. Compared with its sulfoxide derivative B2, the pyrolysis of B1 is significantly faster and more complete under the reaction conditions. The decomposition product B9 also proves to be a good ligand for the arylation reaction, giving the product in a slightly lower yield under the identical reaction conditions.

The materials and methods of the appended claims are not limited in scope by the specific materials and methods described herein, which are intended as illustrations of a few aspects of the claims and any materials and methods that are functionally equivalent are within the scope of this disclosure. Various modifications of the materials and methods in addition to those shown and described herein are intended to fall within the scope of the appended claims. Further, while only certain representative materials, methods, and aspects of these materials and methods are specifically described, other materials and methods and combinations of various features of the materials and methods are intended to fall within the scope of the appended claims, even if not specifically recited. Thus a combination of steps, elements, components, or constituents can be explicitly mentioned herein; however, all other combinations of steps, elements, components, and constituents are included, even though not explicitly stated.

What is claimed is:

1. A method of functionalizing a carbonyl compound at a β-carbon with an aryl or heteroaryl group, comprising: contacting a carbonyl compound comprising a β carbon with an aryl or heteroaryl halide, aryl or heteroaryl tosylate, aryl or heteroaryl triflate, or diaryliodonium salt; a palladium catalyst, a ligand, and a promoter, thereby producing arβ-aryl or heteroaryl carbonyl compound.

2. The method of claim 1, wherein the carbonyl compound is a linear compound having Formula I: 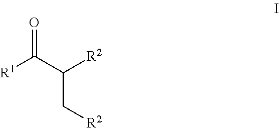
wherein
R¹ is OR³, N(R³)₂, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups;
n is 0, 1, 2, 3, 4, or 5;
each R² is, independent of any other, H, OH, SH, C(O)H, CO₂H, C(O)R³, CO₂R³, OR³, NO₂, CN, N(R³)₂, NC(O)R³, C(O)N(R³)₂, CNR³, Si(R³)₃, SO₂R³, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups; or
two R² groups are on adjacent carbons and together form a fused cycloalkyl ring, cycloheteroalkyl ring, aryl, or heteroaryl ring optionally substituted with one or more additional R² groups;
D is a bond, O, S, NR³, or —(C(R³)₂)₁₋₃; and
each R³ is, independent of any other, H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups.

3. The method of claim 1, wherein the carbonyl compound is a cyclic compound having Formula II: 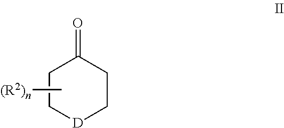
wherein
R¹ is OR³, N(R³)₂, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups;
n is 0, 1, 2, 3, 4, or 5;
each R² is, independent of any other, OH, SH, C(O)H, CO₂H, C(O)R³, CO₂R³, OR³, NO₂, CN, N(R³)₂, NC(O)R³, C(O)N(R³)₂, CNR³, Si(R³)₃, SO₂R³, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups; or
two R² groups are on adjacent carbons and together form a fused cycloalkyl ring, cycloheteroalkyl ring, aryl, or heteroaryl ring optionally substituted with one or more additional R² groups;
D is a bond, O, S, NR³, or —(C(R³)₂)₁₋₃; and
each R³ is, independent of any other, H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups.

4. The method of claim 3, wherein the carbonyl compound has Formula III: 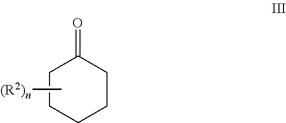
wherein
n is 0, 1, 2, 3, 4, or 5; and
each R² is, independent of any other, OH, SH, C(O)H, CO₂H, C(O)R³, CO2R³, OR³, NO₂, CN, N(R³)₂, NC(O)R³, C(O)N(R³)₂, CNR³, Si(R³)₃, SO₂R³, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups; or
two R² groups are on adjacent carbons and together form a fused cycloalkyl ring.

5. The method of claim 3, wherein n is 1.

6. The method of claim 3, where R² is substituted or unsubstituted C₁₋₂₀ alkyl, substituted or unsubstituted aryl, or substituted or unsubstituted heteroaryl.

7. The method of claim 1, wherein the carbonyl compound is cyclohexanone.

8. The method of claim 1, wherein the aryl or heteroaryl halide is an aryl or heteroaryl iodide or aryl or heteroaryl bromide.

9. The method of claim 1, wherein the aryl or heteroaryl halide, aryl or heteroaryl tosylate, or aryl or heteroaryl triflate are used and have Formula IV: 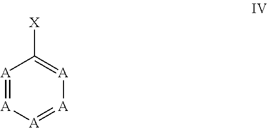
wherein
X is Cl, Br, I, OTs, or OTf;
each A is, independent of the others, N, CH, or CR⁴;
each R⁴ is, independent of the others, F, Cl, Br, I, OH, SH, C(O)H, CO₂H, C(O)R⁵, CO₂R⁵, OR⁵, NO₂, CN, N(R⁵)₂, NC(O)R⁵, C(O)N(R⁵)₂, CNR⁵, Si(R⁵)₃, SO₂R⁵, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups; or
two R⁴ groups are on adjacent carbons and together form a fused cycloalkyl ring, cycloheteroalkyl ring, aryl, or heteroaryl ring optionally substituted with one or more additional R⁴ groups; and
each R⁵ is, independent of any other, H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups.

10. The method of claim 1, wherein the aryl halide, aryl tosylate or aryl triflate are used and have Formula V: 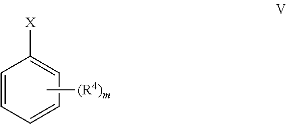
wherein
X is Cl, Br, I, OTs, or OTf;
m is 0, 1, 2, 3, 4, or 5; and
each R⁴ is, independent of the others, F, Cl, Br, I, OH, SH, C(O)H, CO₂H, C(O)R⁵, CO₂R⁵, OR⁵, NO₂, CN, N(R⁵)₂, NC(O)R⁵, C(O)N(R⁵)₂, CNR⁵, Si(R⁵)₃, SO₂R⁵, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups; or
two R⁴ groups are on adjacent carbons and together form a fused cycloalkyl ring, cycloheteroalkyl ring, aryl, or heteroaryl ring optionally substituted with one or more additional R⁴ groups; and
each R⁵ is, independent of any other, H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups.s defined above.

11. The method of claim 10, wherein where X is Br or I.

12. The method of claim 10, wherein m is 2 or 3.

13. The method of claim 10, wherein R⁴ is F, Cl, Br, I, OH, SH, C(O)H, CO₂H, C(O)R⁵, CO₂R⁵, OR⁵, NO₂, CN, N(R⁵)₂, NC(O)R⁵, C(O)N(R⁵)₂, CNR⁵, Si(R⁵)₃, SO₂R⁵, substituted or unsubstituted C₁₋₂₀ alkyl, or substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups.

14. The method of claim 1, wherein the diaryliodonium salt is used and has Formula VI: 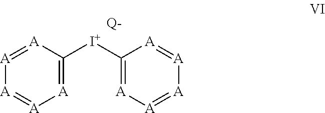
wherein
Q is a counter anion;
each A is, independent of the others, N, CH, or CR⁴;
each R⁴ is, independent of the others, F, Cl, Br, I, OH, SH, C(O)H, CO₂H, C(O)R⁵, CO₂R⁵, OR⁵, NO₂, CN, N(R⁵)₂, NC(O)R⁵, C(O)N(R⁵)₂, CNR⁵, Si(R⁵)₃, SO₂R⁵, substituted or unsubstituted C₁₋₂₀ alkyl, substituted or unsubstituted C₂₋₂₀ alkenyl, substituted or unsubstituted C_{b 2-20} alkynyl, substituted or unsubstituted C₁₋₂₀ eteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups; or
two R⁴ groups are on adjacent carbons and together form a fused cycloalkyl ring, cycloheteroalkyl ring, aryl, or heteroaryl ring optionally substituted with one or more additional R⁴ groups; and
each R⁵ is, independent of any other, H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups.

15. The method of claim 1, wherein the diaryliodonium salt is used and has Formula VII: 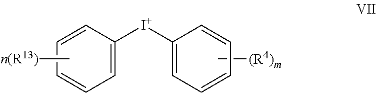
wherein Q is a counter anion;
m is 0, 1, 2, 3, 4, or 5;
n is 0, 1, 2, 3, 4, or 5;
each R⁴ is, independent of the others, F, Cl, Br, I, OH, SH, C(O)H, CO₂H, C(O)R⁵, CO₂R⁵, OR⁵ , NO₂, CN, N(R⁵)₂, NC(O)R⁵, C(O)N(R⁵)₂, CNR⁵, Si(R⁵)₃, SO₂R⁵, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups; or
two R⁴ groups are on adjacent carbons and together form a fused cycloalkyl ring, cycloheteroalkyl ring, aryl, or heteroaryl ring optionally substituted with one or more additional R⁴ groups; and
each R¹³ is H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups.

16. The method of claim 1, wherein stoichiometric amounts of the aryl or heteroaryl halide, aryl or heteroaryl tosylate, aryl or heteroaryl triflate, or diaryliodonium salt and carbonyl compound are used.

17. The method of claim 1, wherein the palladium catalyst comprises palladium(II).

18. The method of claim 1, wherein the palladium catalyst comprises palladium(0).

19. The method of claim 1, wherein the palladium catalyst comprises one or more of palladium(II)chloride, palladium(II)bromide, palladium(II)iodide, palladium(II)acetate, or palladium(II)propionate, palladium(II)trifluoromethanesulfonate, palladium(II)tetrafluoroborate, palladium(II)hexafluorophosphate, or palladium(II)hexafluoroantimonate.

20. The method of claim 1, wherein the palladium catalyst comprises palladium(II)trifluoroacetate.

21. The method of claim 1, wherein the ligand is an organophosphine having formula R⁶R⁷R⁸P, where R⁶, R⁷, and R⁸ are each, independent of one another, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups.

22. The method of claim 1, wherein the ligand is triisopropyl phosphine, trimethyl phosphine, tricyclohexyl phosphine, or mixtures thereof.

23. The method of claim 1, wherein the ligand is a sulfonate, sulfinimide, sulfone, sulfoxide, or sulfide containing ligand.

24. The method of claim 1, wherein the ligand is selected from the group consisting of:

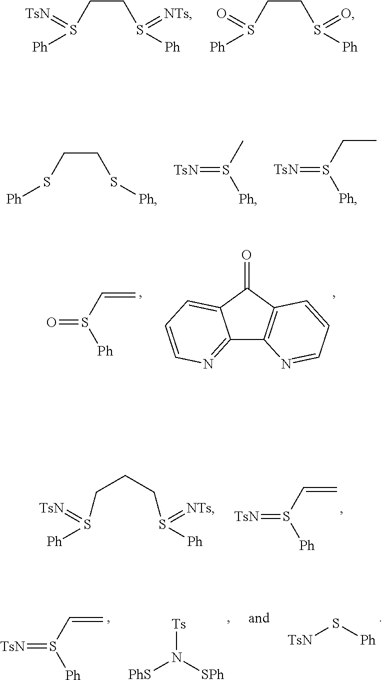

25. The method of claim 1, wherein the ligand is

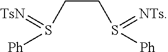

26. The method of claim 1, wherein the promoter comprises a silver, copper, potassium, sodium, lithium, magnesium, or cesium containing compound.

27. The method of claim 1, wherein the promoter comprises silver carbonate, silver sulfate, silver nitrate, silver formate, silver acetate, silver proprionate, silver nitrate, silver chloride, silver bromide, silver iodide, silver trifluoromethanesulfonate, silver tetrafluoroborate, silver hexafluorophosphate, silver hexafluoroantimonate, or any mixture thereof.

28. The method of claim 1, wherein the promoter is silver trifluoroacetate.

29. The method of claim 1, wherein the promoter is promoter is copper trifluoroacetate or potassium trifluoroacetate.

30. There method of claim 1, further comprising a solvent.

31. The method of claim 30, wherein the solvent comprises one or more of 1,4-dioxane, hexafluoroisopropanol or a mixture thereof.

32. A method of functionalizing a carbonyl compound at a β-carbon with an aryl group, comprising: contacting a carbonyl compound having Formula II: 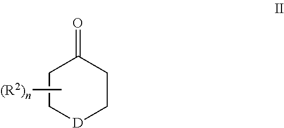
wherein
R¹ is OR³, N(R³)₂, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups;
n is 0, 1, 2, 3, 4, or 5;
each R² is, independent of any other, OH, SH, C(O)H, CO₂H, C(O)R³, CO₂R³, OR³, NO₂, CN, N(R³)₂, NC(O)R³, C(O)N(R³)₂, CNR³, Si(R³)₃, SO₂R³, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups; or
two R² groups are on adjacent carbons and together form a fused cycloalkyl ring, cycloheteroalkyl ring, aryl, or heteroaryl ring optionally substituted with one or more additional R² groups;
D is a bond, O, S, NR³, or —(C(R³)₂)₁₋₃; and
each R³ is, independent of any other, H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups;
with an aryl or heteroaryl halide, aryl or heteroaryl tosylate, or aryl or heteroaryl trilfate having Formula IV 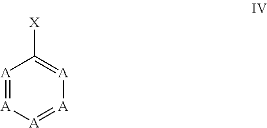
where X is Cl, Br, I, OTs, or OTf;
each A is, independent of the others, N, CH, or CR⁴;
each R⁴ is, independent of the others, F, Cl, Br, I, OH, SH, C(O)H, CO₂H, C(O)R⁵, CO₂R⁵, OR⁵, NO₂, CN, N(R⁵)₂, NC(O)R⁵, C(O)N(R⁵)₂, CNR⁵, Si(R⁵)₃, SO₂R⁵, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups; or
two R⁴ groups are on adjacent carbons and together form a fused cycloalkyl ring, cycloheteroalkyl ring, aryl, or heteroaryl ring optionally substituted with one or more additional R⁴ groups; and
each R⁵ is, independent of any other, H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups,
in the presence of a palladium(II) or palladium (0) catalyst, an organophosphine ligand selected from the group consisting of triisopropyl phosphine, trimethyl phosphine, tricyclohexyl phosphine, and mixtures thereof, and a promoter selected from the group consisting of silver, copper, potassium, sodium, lithium, or cesium trifluoroacetate, to thereby provide a β-aryl carbonyl compound having Formula II-P

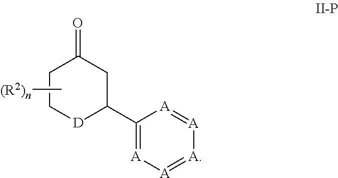

33. A method of functionalizing a carbonyl compound at a β-carbon with an aryl group, comprising: contacting a carbonyl compound having Formula II: 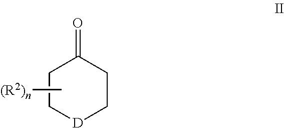
wherein
R¹ is OR³, N(R³)₂, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups;
n is 0, 1, 2, 3, 4, or 5;
each R² is, independent of any other, OH, SH, C(O)H, CO₂H, C(O)R³, CO₂R³, OR³, NO₂, CN, N(R³)₂, NC(O)R³, C(O)N(R³)₂, CNR³, Si(R³)₃, SO₂R³, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups; or
two R² groups are on adjacent carbons and together form a fused cycloalkyl ring, cycloheteroalkyl ring, aryl, or heteroaryl ring optionally substituted with one or more additional R² groups;
D is a bond, O, S, NR³, or —(C(R³)₂)₁₋₃; and
each R³ is, independent of any other, H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups;
with a diaryliodonium salt having Formula VI 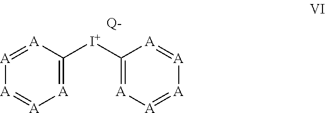
wherein
Q is a counter anion;
each A is, independent of the others, N, CH, or CR⁴;
each R⁴ is, independent of the others, F, Cl, Br, I, OH, SH, C(O)H, CO₂H, C(O)R⁵, CO₂R⁵, OR⁵, NO₂, CN, N(R⁵)₂, NC(O)R⁵, C(O)N(R⁵)₂, CNR⁵, Si(R⁵)₃, SO₂R⁵, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups; or
two R⁴ groups are on adjacent carbons and together form a fused cycloalkyl ring, cycloheteroalkyl ring, aryl, or heteroaryl ring optionally substituted with one or more additional R⁴ groups; and
each R⁵ is, independent of any other, H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₁₋₂₀ heteroalkyl, substituted or unsubstituted C₂₋₂₀ heteroalkenyl, substituted or unsubstituted C₂₋₂₀ heteroalkynyl, substituted or unsubstituted cycloalkyl, substituted or unsubstituted heterocycloalkyl, substituted or unsubstituted aryl, substituted or unsubstituted heteroaryl, or mixtures thereof, wherein any of the substituted groups named can be substituted with one or more of alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol groups,
in the presence of a palladium(II) or palladium (0) catalyst, a bis-sulfinimide ligand, and a promoter selected from the group consisting of copper, potassium, sodium, lithium, magnesium, or cesium trifluoroacetate, to thereby provide a β-aryl carbonyl compound having Formula II-P [chemical structure]
